(19) United States
(12) Patent Application Publication
(10) Pub. No.: US 2013/0274731 A1
(43) Pub. Date: Oct. 17, 2013

(54) HELICAL TUBING DEVICES AND METHODS FOR FLUID RENAL NERVE MODULATION

(71) Applicant: BOSTON SCIENTIFIC SCIMED, INC., Maple Grove, MN (US)

(72) Inventors: JAMES M. ANDERSON, FRIDLEY, MN (US); DEREK C. SUTERMEISTER, HAM LAKE, MN (US)

(73) Assignee: BOSTON SCIENTIFIC SCIMED, INC., MAPLE GROVE, MN (US)

(21) Appl. No.: 13/864,136

(22) Filed: Apr. 16, 2013

Related U.S. Application Data
(60) Provisional application No. 61/624,944, filed on Apr. 16, 2012.

(51) Int. Cl. A61B 18/14; A61B 18/18

(52) U.S. Cl. CPC A61B 18/1492; A61B 18/18
USPC 606/33; 606/41

(57) ABSTRACT

Medical devices and methods for making and using medical devices are disclosed. An example medical device may include a catheter shaft having a proximal end, a distal portion, and a lumen formed therein. The distal portion may be capable of shifting between a first configuration and a second helical configuration. The distal portion may have a plurality of fluid ports formed therein. An electrode may be disposed within the lumen. The electrode may be capable of transmitting energy through the plurality of fluid ports.

### CROSS-REFERENCE TO RELATED APPLICATIONS

This application claims priority under 35 U.S.C. §119 to U.S. Provisional Application Ser. No. 61/624,944, filed Apr. 16, 2012, the entirety of which is incorporated herein by reference.

### FIELD

The invention generally pertains to percutaneous and intravascular devices for nerve modulation and/or ablation.

### BACKGROUND

Certain treatments require the temporary or permanent interruption or modification of select nerve function. One example treatment is renal nerve ablation which is sometimes used to treat conditions related to congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many body tissues such as nerves, including renal nerves, brain tissue, cardiac tissue and the tissue of other body organs are in close proximity to blood vessels or other body cavities and thus can be accessed percutaneously or intravascularly through the walls of the blood vessels. In some instances, it may be desirable to ablate perivascular nerves using a radio frequency (RF) electrode. In other instances, the perivascular nerves may be ablated by other means including application of thermal, ultrasonic, laser, microwave, and other related energy sources to the vessel wall.

In treatments involving perivascular nerves such as renal nerves, treatment methods employing such energy sources have tended to apply the energy as a generally circumferential ring to ensure that the nerves are modulated. However, such a treatment may result in thermal injury to the vessel wall near the electrode and other undesirable side effects such as, but not limited to, blood damage, clotting, weakened vessel wall, and/or protein fouling of the electrode.

### SUMMARY

It is therefore desirable to provide for alternative systems and methods for tissue treatment such as intravascular nerve modulation treatments that distribute ablation or modulation sites along and around the vessel or other body cavity.

Some embodiments of the invention are directed to a therapeutic catheter configured for tissue modulation such as nerve modulation and/or nerve ablation and are suited for use in a body lumen such as an artery. The therapeutic catheter includes a lumen to allow fluid flow from a fluid source (such as a syringe attached to a proximal hub) to a distal section of the catheter. The distal section of the catheter includes a number of ports in the side wall of the catheter to allow the fluid to exit the catheter. The distal end of the catheter may be capped or otherwise closed. An electrode extending through the distal section is electrically connected to a power supply. When the electrode is activated with for, example, a radio-frequency (RF) current, the fluid flowing from the ports in the distal section transmits the therapeutic effect of the current from the electrode to the walls of the body lumen. The electrode may be a substantially straight wire, a coil, a braid or other suitable configuration extending through the distal section. The distal section is further preferably biased to a helical or spiral configuration, and the ports in the distal section are preferably arranged to be directed towards the lumen wall, either directly radially outward or at an angle, when the distal section is in the helical configuration. The distal section is flexible enough that it may be collapsed into a straight configuration when withdrawn into a guide catheter. The ports are preferably arranged to provide complete circumferential coverage of the wall of the vessel lumen while each treatment area associated with a particular port is spaced longitudinally from each other.

Some embodiments pertain to a method of using a therapeutic catheter as described above, where such a catheter is introduced percutaneously into a body lumen such as a renal artery to a desired location. When the therapeutic catheter distal section is in the expanded helical configuration, a fluid such as saline is introduced through the lumen and out the distal ports and RF energy is supplied to the electrode to effect an ablation therapy. The therapy is continued for an effective amount of time such as one minute or more. The catheter may be repositioned and the procedure repeated to effect the therapy at a second location.

The above summary of some example embodiments is not intended to describe each disclosed embodiment or every implementation of the invention.

### BRIEF DESCRIPTION OF DRAWINGS

The invention may be more completely understood in consideration of the following detailed description of various embodiments in connection with the accompanying drawings, in which:

FIG. 1 is a schematic view illustrating a renal nerve modulation system in situ.

FIG. 2 is an isometric view illustrating the distal section of a renal nerve modulation system.

FIG. 3 is a diagrammatic view illustrating the distal section of a renal nerve modulation system.

FIG. 4 is a diagrammatic view illustrating the distal section of a renal nerve modulation system.

FIGS. 5a and 5b are diagrammatic views illustrating a portion of distal section of renal nerve modulation systems.

FIG. 6 is a diagrammatic view of a distal section of a renal nerve modulation system in situ.

While embodiments of the present disclosure are amenable to various modifications and alternative forms, specifics thereof have been shown by way of example in the drawings and will be described in detail. It should be understood, however, that the intention is not to limit aspects of the disclosure to the particular embodiments described. One the contrary, the intention is to cover all modifications, equivalents, and alternatives falling within the spirit and scope of the present disclosure.

### DETAILED DESCRIPTION

For the following defined terms, these definitions shall be applied, unless a different definition is provided in the claims or elsewhere in the specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant numbers.

The recitation of numerical ranges by endpoints includes all numbers within that range (e.g., 1 to 5 includes 1, 1.5, 2, 2.75, 3, 3.80, 4, and 5).

Although some suitable dimension ranges and/or values pertaining to various components, features, and/or specifications are disclosed, one of skill in the art, incited by the present disclosure, would understand desired dimensions, ranges and/or values many deviate from those expressly disclosed.

As used in this specification and the appended claims, the singular forms “a,” “an,” and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

While the devices and methods described herein are discussed relative to renal nerve modulation, it is contemplated that the devices and methods may be used in other applications where nerve modulation and/or ablation are desired. For example, the device and methods may be used in any other blood vessels or body lumen where nerve modulation or other tissue modulation is desired.

In some instances, it may be desirable to ablate perivascular renal nerves with deep target tissue heating. However, as energy passes from an electrode to the desired treatment region, the energy may heat the fluid (e.g. blood) and tissue as it passes. As more energy is used, higher temperatures in the desired treatment region may be achieved, but may result in some negative side effects, such as, but not limited to, thermal injury to the blood vessel wall, damage to or clotting of the blood cells themselves, and/or fouling the electrode. Positioning the electrode away from the blood vessel wall may provide some degree of passive cooling by allowing blood to flow past the electrode. Further, in embodiments described herein, the electrical energy providing the ablation treatment is provided through the vessel wall by a fluid. This fluid may provide further cooling to the vessel wall.

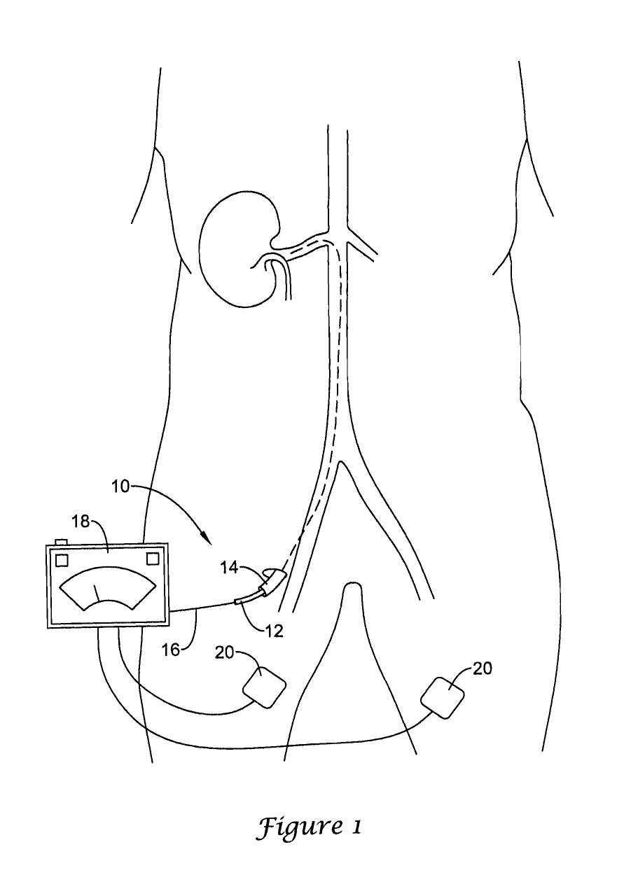

FIG. 1 is a schematic view of an illustrative renal nerve modulation system 10 in situ. System 10 may include a catheter 12 introduced percutaneously into the body by a guide catheter 14 or other means known in the art. A power conductor such as a wire 16 connects the catheter 12 to a control unit and power supply 18, which supplies the necessary electrical energy to activate the one or more electrodes (not shown) in a distal section of the catheter 12. In some instances, return electrode patches 20 may be supplied on the legs, abdomen, or at another conventional location on the patient's body to complete the circuit. In other instances, the return electrode patches 20 may not be necessary. For example, the catheter 12 may include one or more pairs of bipolar electrodes. The control unit and power supply 18 may include monitoring elements to monitor parameters such as power, temperature, voltage, pulse size and/or shape and other suitable parameters as well as suitable controls for performing the desired procedure, and corresponding connections to the catheter. In some instances, the control unit and power supply 18 may control a radio frequency (RF) electrode. The electrode may be configured to operate at a frequency of about 460 kHz. It is contemplated that any desired frequency in the RF range may be used, for example, from about 450 to about 500 kHz. However, it is also contemplated that different types of energy outside the RF spectrum may be used as desired. The system may include other elements, not illustrated, such as a guidewire. The system also includes a source of fluid that may be attached to the catheter using a conventional catheter hub with luer ports or the like. The fluid sources may be syringes or may include electronically controlled pumps to control the rate or volume of fluid introduced.

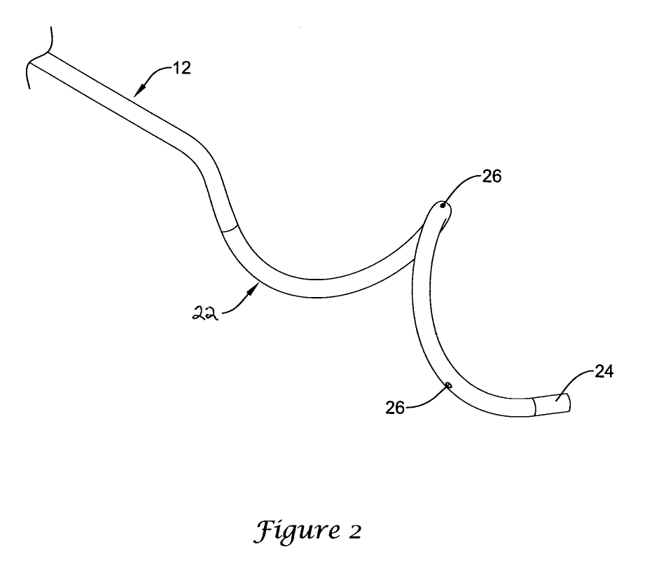

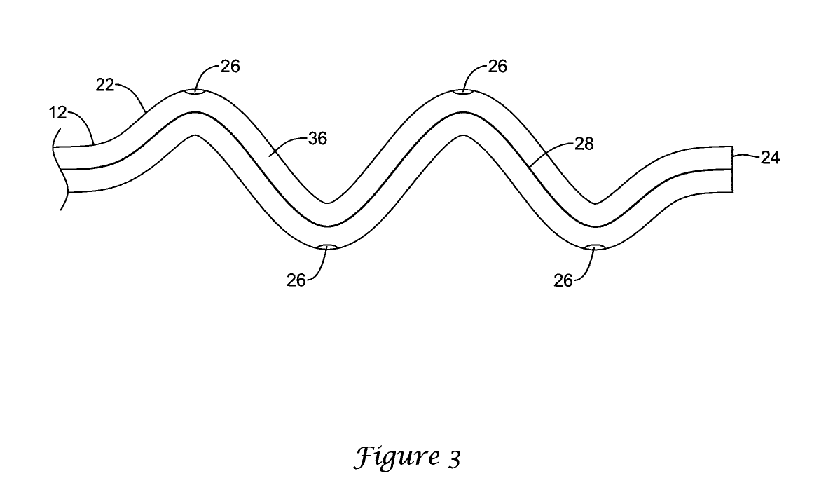

FIGS. 2 and 3 are isometric and diagrammatic views, respectively, of a distal end portion 22 of a catheter 12 of an illustrative renal nerve modulation system 10. The distal end portion 22 includes a lumen 36 extending proximally through the catheter 12 to a connection to a fluid source. The connection to the fluid source may be at the proximal end of the catheter 12 or may be at another proximal location. The lumen 36 may be closed or otherwise terminates at the distal end 24 of the catheter 12. Side ports 26 in the distal end section are fluidly connected to the lumen 36. The catheter 12 is flexible and the catheter may be withdrawn into a guide catheter 14 or the like to collapse the catheter 12 into a substantially straight configuration. When not confined by a guide catheter 14 or the like, the distal end portion 22 may expand to a helical shape as illustrated. The helical shape may be a smooth helix as shown or may include straight sections between the side ports 26. In such a configuration the side ports 26 may be on elbows between the straight sections or may be on other straight sections. Any arrangement by which the ports 26 are arranged as described herein may be suitable. When the distal end portion 22 is in the expanded configuration, the side ports 26 have a radially outwardly facing component. This means that any fluid ejected through the side ports 26 is ejected along vectors that have a radial component and where the directions of those vectors is outward from the central longitudinal axis of the catheter. It will be appreciated that when the distal end portion 22 is in the expanded, helical state, the distal portion circles but does not necessarily contact the central longitudinal axis of the catheter 12. In some embodiments, the side ports 26 face directly outwardly such that the fluid vector is coincident with a radius from the central longitudinal axis. In other embodiments, the side ports 26 are at an angle to the radius such that only a component of the vector is coincident with a radius. In such embodiments, the side ports may be at an angle of between 0 and 90 degrees (exclusive), of between 0 and 45 degrees, of between 5 and 40 degrees, of between 10 and 35 degrees, of between 15 and 30 or at another suitable angle to a radius from the central longitudinal axis. The side ports 26 are distributed along the distal end portion 22.

The distal end portion 22 may be biased to the expanded helical configuration and may naturally return to such a configuration when any constraint is removed. In other embodiments, the distal end portion may be biased to a straight configuration and may be moved to the helical configuration by action of a pull wire or the like (not illustrated). Such a pull wire may be disposed within lumen 36 or external to the distal end portion 22.

The catheter 12 and in particular the distal end portion 22 may have a typical cylindrical cross section or may have another suitable cross section. For example, in some embodiments, the cross-section of the distal portion may be oval or oblong. In such embodiments, the distal end portion 22 may be arranged like a typical helical ribbon wire, with the wider portion forming the outer (and inner) surface of the helix. Other suitable cross-sectional profiles may include polygonal profiles such as hexagonal or octagonal.

In some embodiments, the side ports 26 are disposed along the distal end portion 22 so as to provide a complete circumferential coverage of the lumen wall to be treated. Thus, for example, if each side port 26 can create a therapeutic effect over a 6 mm long portion of the artery wall, and the catheter 12 is designed to be used with an artery having a 15 mm circumference on its inner wall, then three side ports 26 are indicated. The three side ports 26 will be disposed to cover a different arc of the vessel wall, and will be spaced longitudinally from each other so that the three treatment areas do not overlap longitudinally. The treatment areas may overlap circumferentially such that the same circumferential portion of the vessel wall may be treated by more than one side port 26. These are just examples.

Any suitable number of side ports 26 may be included, and it will be observed that the number of side ports 26 that may be appropriate is a function of the diameter of the vessel to be treated, the effective treatment area of a side port 26 and the degree of overlap desired. In some embodiments, a further degree of redundancy may be provided where the distal end portion 22 extends for more than one helical loop (such as two or three helical loops) and the side ports 26 extend along the distal end portion 22 to ensure that any particular circumferential section is treated at two, three or more longitudinally spaced locations.

Operation of the system may be between understood with reference to FIG. 3, which is a diagrammatic view of the distal end portion 22 of catheter 12 illustrating the inside of the catheter 12. An electrode 28 extends through a lumen 36, which lumen 36 is fluidly connected to the fluid supply discussed above and to the side ports 26. During use the electrode 28 may be activated with RF energy and fluid such as saline may be supplied by the fluid source and exit through the side ports 26. The fluid provides a conductive path for the RF energy and may also provide cooling to the intima and perhaps some of the media of the vessel wall. The RF energy ablates or modulates the tissue, preferably destroying nerve tissue located in the adventitia, while the cooling effect of the saline (and the blood) prevents any such modulation of the intima and media.

The electrode 28 illustrated in FIG. 3 may take the form of a wire that extends along the distal end portion 22 of the catheter 12 and loosely follows the path of the distal end portion 22. The electrode 28 may extend to the distal end 24 of the catheter 12 or may simply extend under the side ports 26. The electrode 28 may be made from any suitable conductive material such as stainless steel or another metal. Further, the electrode 28 may at least in part provide the distal end portion 22 of the catheter 12 with the structure needed to expand into the unconstrained shape. Electrode 28 is electrically connected through wire 16 to the control unit and power supply 18. Electrode 28 may be a simple wire, having a circular cross-sectional area or may be a ribbon wire or other desired shape.

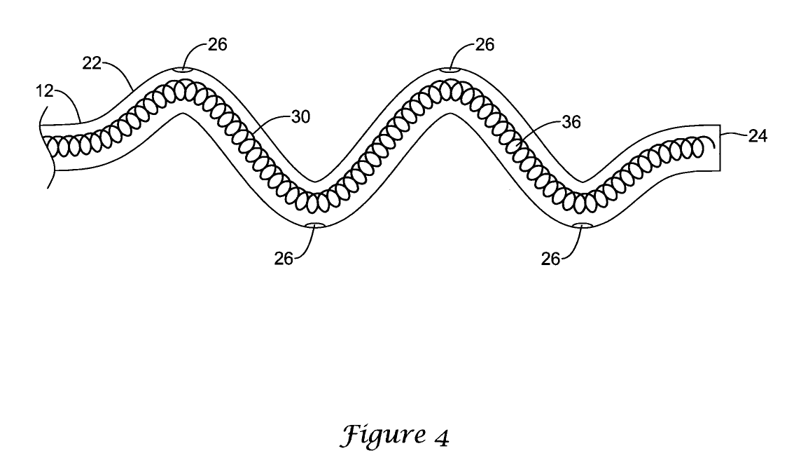

FIG. 4 diagrammatically illustrates an embodiment in which the electrode is a coil electrode 30. The coil electrode 30 may be used in place of or in conjunction with other electrodes disclosed herein. In some embodiments, the pitch of the coil electrode 30 may vary along the length of the catheter 12 (e.g., the pitch may be tighter along the distal portion 22).

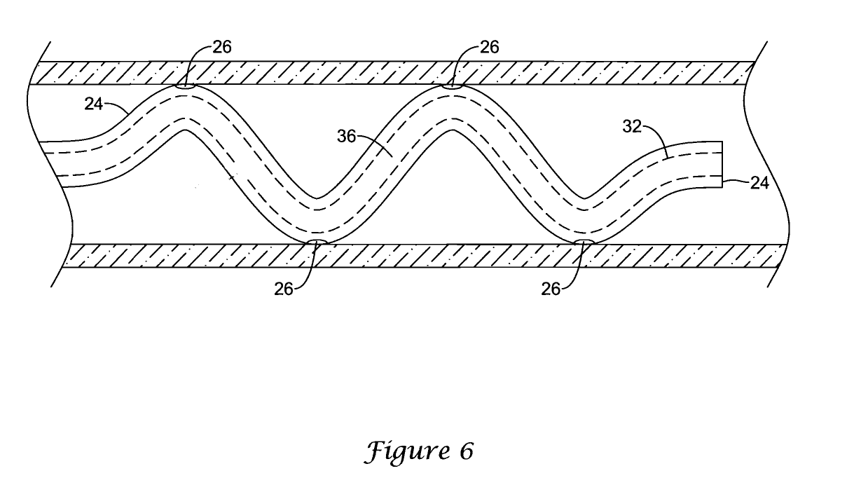

In the embodiment illustrated in FIG. 6, the electrode is a braid electrode 32. The braid electrode 22 may be used in place of or in conjunction with other electrodes disclosed herein. Braid electrode 32 may be an element of catheter 12 and may consequently act as a typical braid reinforcement member as well as an electrode. For example, the catheter 12 may have a wall and the braid electrode 32 may be disposed along or embedded within the wall. In some embodiments, braid electrode 32 may be exposed to the lumen 36, having no intervening layers. In other embodiments, an electrically conductive liner may be disposed between the braid electrode 32 and the lumen 36. Polytetrafluoroethylene (ptfe) may be a suitable material for such a liner.

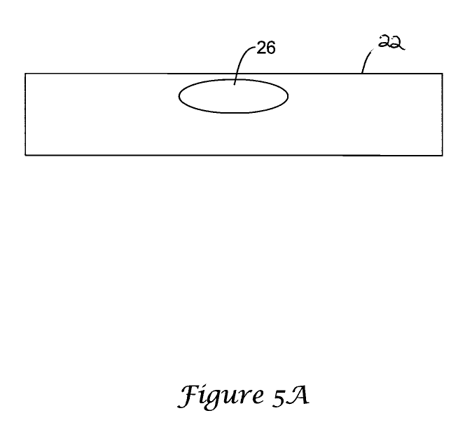

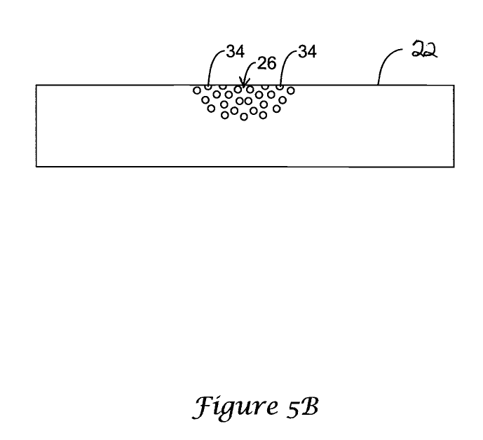

FIGS. 5A and 5B are short sections of the distal end portion 22 illustrating with greater detail the configuration of a side port 26. In FIG. 5A, the side port 26 is a single lumen port. The side ports 26 may be circular, oval or other suitable shape. If the side ports are circular, a suitable diameter may be between 0.1 and 1 mm. In FIG. 5B, the side port 26 (which may be described as a side port region 26) is comprised of a number of micro-openings 34. Each micro-opening may be less than 40 microns, less than 30 microns, less than 25 microns, between 10 and 25 microns or another suitable size. The combined area of the micro-openings 34 in a particular side port 26 may be between 0.1 mm and 1 mm. In embodiments that include a braid electrode 32, each of the micro-openings 34 may be located within an interstice of the braid. Any of the embodiments described herein may include either single lumen side ports as shown in FIG. 5A or side ports 26 comprised of micro-openings as shown in FIG. 5B. It is contemplated that some embodiments will have only one style of side port 26.

Catheter 12 may be made of one or more of any suitable biocompatible material such as a polymeric, or any other such material for example, a polymeric, electrically nonconductive material, such as polyethylene, polyurethane, or PEBAX® material (polyurethane and nylon). In addition, the distal end portion 22 may be made more flexible than a proximal portion by using different material and/or having a thinner wall thickness.

The conductive fluid ablates the vessel wall by transferring radio frequency electrical current from the electrode to the vessel wall. In general, the conductive fluid acts as a conducting medium to transfer radio frequency electrical current. The conductive fluid may generally be a water soluble, biocompatible, non-toxic, and electrically conductive fluid. Suitable fluids that may be used as the conductive fluid include salines such as isotonic saline and the like. In addition, a quantity of radiopaque fluid may be used as well. The radiopaque fluid may be mixed with the conductive fluid to provide for constant visualization or may be introduced periodically and discretely through the fluid channels to provide for visualization at discrete intervals.

In use, the system 10 may be used for ablating a renal nerve through a blood vessel lumen, which may facilitate in treatment of conditions related to congestive heart failure. Referring to FIG. 6, an operator may insert the system 10 within a renal artery. When at the desired location, a guide catheter 14 may be withdrawn proximally from catheter 12 to allow it to expand to a helical configuration as shown. The operator may then initiate saline flow through the lumen 36 and out side ports 26 and activate the electrode 32 (e.g., the braid electrode 32 as shown in FIG. 6 and/or other electrodes including those disclosed herein). RF energy at a frequency of between 400 and 500 KHz and preferably about 450 KHz is supplied through the electrode 32 (e.g., the braid electrode 32 and/or other electrodes including those disclosed herein) to the vessel wall. The power level may be between 4 and 40 watts, between 5 and 8 watts or another desired level. The fluid provides a conductive pathway from the electrode to the vessel wall. The rate of fluid flow through the side ports may be increased to increase the size of the effective area or decreased to reduce the size of the effective area. A suitable flow rate may be between 2 ml/min and 20 ml/min. The treatment may be continued for an effective time; in some cases an effective time is about 1.5 minutes. Conditions may be monitored through the use of a thermocouple or thermister at or near one of the side ports. The progress of the treatment may be monitored by monitoring the impedance of the RF circuit as the impedance may change as the condition of the tissue changes. At the end of a treatment, the catheter 12 may be repositioned and another treatment initiated. Removal of the catheter may be effected by withdrawing the catheter 12 into a catheter sheath such as guide catheter 14 to collapse the distal portion and then withdrawing the system 10 from the patient.

Those skilled in the art will recognize that the present disclosure may be manifested in a variety of forms other than the specific embodiments described and contemplated herein. Accordingly, departure in forma and detail may be made without departing from the scope and spirit of the present disclosure as described in the appended claims.

What is claimed is:

1. A medical device, comprising:
a catheter shaft having a proximal end, a distal portion, and a lumen formed therein;
wherein the distal portion is capable of shifting between a first configuration and a second helical configuration;
wherein the distal portion has a plurality of fluid ports formed therein; and
an electrode disposed within the lumen, wherein the electrode is capable of transmitting energy through the plurality of fluid ports.

2. The medical device of claim 1, wherein the catheter shaft has a closed distal end.

3. The medical device of claim 1, wherein the electrode is capable of transmitting energy to a conductive fluid disposed within the lumen and through the plurality of fluid ports.

4. The medical device of claim 1, wherein the plurality of fluid ports face radially outwardly when the distal portion of the catheter shaft is unconstrained.

5. The medical device of claim 1, wherein each of the plurality of fluid ports has a maximum width of between 30 and 300 μm.

6. The medical device of claim 1, wherein each of the plurality of fluid ports comprises a micro-opening.

7. The medical device of claim 6, wherein at least some of the micro-openings have a maximum width of less than 30 microns.

8. The medical device of claim 6, wherein at least some of the micro-openings have a maximum width of less than 25 microns.

9. The medical device of claim 6, wherein the micro-openings are disposed in a circular arrangement.

10. The medical device of claim 1, wherein the electrode extends to a position under each of the plurality of fluid ports.

11. The medical device of claim 1, wherein the electrode includes a wire.

12. The medical device of claim 1, wherein the electrode includes a coil.

13. The medical device of claim 1, wherein the electrode includes a braid.

14. The medical device of claim 13, wherein the plurality of fluid ports include a plurality of micro-openings, wherein the braid has interstitial openings, and wherein the micro-openings are positioned along the interstitial openings.

15. The medical device of claim 13, wherein the catheter shaft has a wall and wherein the braid is disposed within the wall.

16. The medical device of claim 1, wherein the plurality of fluid ports are spaced at regular intervals.

17. The medical device of claim 1, wherein the plurality of fluid ports are helically disposed around the distal portion.

18. A medical device for ablating one or more nerves adjacent to a renal artery, the medical device comprising:
a catheter shaft having a proximal end, a helical distal portion, and a fluid lumen formed therein for passing a conductive fluid therethrough;
wherein the distal portion has a plurality of fluid ports formed therein;
an electrode disposed within the fluid lumen; and
wherein the electrode is capable of conducting energy to the conductive fluid and through the plurality of fluid ports.

19. The medical device of claim 18, wherein the electrode includes an RF electrode.

20. A method of nerve modulation, the method comprising:
providing a medical device, comprising:
a catheter shaft having a proximal end, a helical distal portion, and a fluid lumen formed therein for passing a conductive fluid therethrough,
wherein the distal portion has a plurality of fluid ports formed therein;
an electrode disposed within the fluid lumen, and
wherein the electrode is capable of conducting energy to the conductive fluid and through the plurality of fluid ports;
advancing the medical device through a blood vessel to a position adjacent to a renal artery;
activating the electrode; and
infusing the conductive fluid through the fluid lumen and through the plurality of fluid ports.
